(19) United States
(12) Patent Application Publication
(10) Pub. No.: US 2010/0048644 A1
(43) Pub. Date: Feb. 25, 2010

(54) USE OF COMPOSITION COMPRISING AMLODIPINE IN PREPARING MEDICAMENTS FOR TREATING A LOWER URINARY TRACT DISORDER

(75) Inventors: Xiping Xu, Hefei (CN); Houxun Xing, Hefei (CN); Xianhui Qin, Beijing (CN); Haipeng Liu, Hefei (CN); Mingxia Chen, Hefei (CN); Duo Yu, Beijing (CN); Yan Wang, Beijing (CN); Guangliang Chen, Hefei (CN)

Correspondence Address: SHERIDAN ROSS PC, 1560 BROADWAY, SUITE 1200, DENVER, CO, 80202, US

(73) Assignees: BEIJING HUAANFO BIOMEDICAL RESEARCH CENTRE INC., Beijing (CN); ANHUI MEDICAL UNIVERSITY BIOMEDICAL INSTITUTE, Anhui (CN)

(21) Appl. No.: 12/447,438

(22) PCT Filed: Oct. 25, 2007
(86) PCT No.: PCT/CN2007/003041

§ 371 Date: Jun. 12, 2009

(30) Foreign Application Priority Data
Oct. 25, 2006 (CN) 200610114017.5

(51) Int. Cl. A61K 31/435; A61P 13/02

(52) U.S. Cl. 514/356

(57) ABSTRACT

Use of composition comprising amlodipine in preparing medicaments for treating a lower urinary tract disorder. The lower urinary tract disorder is selected from the group consisting of benign prostate hyperplasia, lower urinary tract syndrome or overactive bladder, especially the lower urinary tract disorder accompanying with hypertension. Amlodipine is any one selected from the group consisting of amlodipine, amlodipine active metabolite, levoamlodipine, pharmaceutically acceptable precursor of amlodipine, or pharmaceutically acceptable salts of amlodipine, amlodipine active metabolite, levoamlodipine, or pharmaceutically acceptable precursor of amlodipine. The pharmaceutical dose of amlodipine is in the range of 1-10 mg. The invention belongs to the field of pharmacy.

### TECHNICAL FIELD

The present invention relates to the usage of pharmaceutical composition comprising Amlodipine for the manufacture of medicaments for treating lower urinary tract disorders, which consist of benign prostate hyperplasia, lower urinary tract syndrome, and overactive bladder.

### BACKGROUND OF THE INVENTION

Benign prostate hyperplasia (BPH) is one of the most common chronic diseases in the middle-aged and elderly men. As the aging populations continue to grow in the world, so do the number of cases of BPH. In China, an epidemiological investigation showed that the prevalence of adult prostate hyperplasia was 50% in men aged 60 years or older in Shanghai (Shi R, Wang Y, Len J. An epidemiological Study on adult BPH in Shanghai City. Journal of Shanghai Second Medical University 1999, 19 (3):27-22). Another Chinese survey of 3361 male subjects aged 60 years or older in six cities found that the overall prevalence of BPH was 44%, and the age-specific prevalence rates were 35%, 40%, 47%, 51%, 57%, and 60% for 60 years-, 65 years-, 70 years-, 75 years-, 80 years-, and 85 years respectively. (Yu P, Zheng H, Su H, et al. The incidence rates and relevant factors of prostate hyperplasia in the aged population in six cities in China. Chinese Journal of Epidemiology, 2000, 21(4):276-279). Prostate hyperplasia usually occurs in two lateral lobes and middle lobe of the prostate gland. The enlarged part may protrude into the bladder and elevate the bladder outlet above the bladder base, resulting in bladder dysfunction in bladder empting. The BPH-induced primary insult is urinary tract obstruction. Not always in direct proportion to the size of enlargement, the degree of obstruction mainly depends on that of oppression by enlarged prostate on urinary tract. The obstructive factors induced by BPH include static and dynamic ones, the former refers to a substantial obstruction caused by enlarged prostate while the latter involves increased tensions of prostate, prostatic envelope and bladder neck. There are abundant α-adrenoceptors in prostate and bladder neck. The physiological and pharmacological studies demonstrate that human prostate cells can stimulate smooth muscle contraction increase tension via α-adrenoceptors, which leads to bladder outflow obstruction (BOO).

In recent years, with emergence of drugs that could control BPH and improve urethral obstruction, physicians and patients are paying more attentions to drug therapy. Current anti-BPH drugs mainly fall into three categories:

1. 5α-reductase inhibitors: such as Finasteride, aiming at BPH pathogenesis and exerting a therapeutical action by inhibiting 5α-reductase, which promotes prostate hyperplasia.
2. α-receptor blockers: such as Terazosin and Tamsulosin, relaxing urinary tract to increase urinary flow by inhibiting muscle contraction of bladder neck to posterior urinary tract, yet failing to reduce prostatic volume.
3. Plant drugs: having an analgesic and anti-inflammatory action, and usually used in the treatment of BPH and prostatitis by relieving symptoms, the pharmacological mechanism of which remains unclear.

Lower urinary tract syndromes (LUTS), a term referring to symptoms occurring during bladder filling (irritating) and/or bladder emptying (obstructive), is common in the elderly (Recommendation of International scientific committee on the 5th International Consultation on BPH: Evaluation and treatment of low urinary tract syndromes in old men: Chinese Journal of Urology 2001, 22:564-570). Irritating symptoms in bladder filling include detrusor instability, bladder hyperaesthesia, reduced bladder volume, urgent and frequent micturition, urinary incontinence, and nocturia, etc. Obstructive symptoms in bladder emptying include dysuria, weak urinary stream, terminal dribbling, urinary retention, etc. Symptoms during bladder filling are caused by both bladder storage dysfunction and abnormal bladder emptying. A large number of studies showed that obstructive dynamic factor is a predominant reason for LUTS.

The pathogenesis of LUTS is multi-factorial, including BPH, prostatitis, prostate cancer, bladder neck spasm, neurogenic bladder, bladder cancer, etc. Pathological change in bladder detrusor itself, such as senile degenerative changes of detrusor, is also one of the causes of “irritating symptoms”. LUTS is common in the general population: 5% of children suffer from nocturnal enuresis; 15% of women and 7% of men have bladder emptying disorders; about 80% of men over 70 years old are suffering from BPH, and more than a half of whom have remarkable prostate enlargement in volume, in which 50% of these enlarged prostates will result in bladder outlet obstruction (BOO). In elderly men, BPH is the most common cause of LUTS.

Overactive bladder (OAB) causes LUTS, especially of bladder irritation during urinary storage. OAB studies are receiving increasing attention. OAB, being ranked as one of top 10 common chronic diseases in U.S., shows a higher incidence rate than those of diabetic mellitus and gastrointestinal ulcer. [Paul Abrams and Alan J Wein: Introduction: Overactive bladder and its treatments Urology 2000, 55: 1]. OAB could be classified as primary and secondary to other diseases. Primary OAB has a clinical course of more than half a year, without a definite etiology. Secondary OAB can be caused by many diseases, mainly including BPH, female bladder neck obstruction, neurogenic bladder emptying dysfunction (for example, caused by stroke, spinal cord injury and Parkinson's disease), local lesion of bladder, impaired contractility of detrusor, etc. The symptoms of OAB usually involve frequent urination, nocturia, and urgent micturition with or without urgent incontinence, of which, urgent micturition occurs most commonly, with an incidence rate of 9.2%. Therapeutic regime of OAB includes drug, diet, bladder training procedure, electrostimulation, and surgical operation. Antimuscarinic agents are commonly used for the treatment of OAB. Yet, this kind of drug has low therapeutic efficacy. In addition, it is associated with side effects, such as dry eye, dry mouth, palpitation, lethargy and constipation, making it intolerable in some patients.

In summary, while BPH, LUTS and OAB are intimately related, they represent independent syndromes or diseases. Clinically, the diagnosis of BPH includes three criteria: 1) Prostate enlargement in volume; 2) Bladder emptying dysfunction, the degree of which may be evaluated by using International Prostate Symptom Score (IPSS); 3) A decreased urinary flow rate. Maximum urinary flow rate is of more valuable than average urinary flow rate. BPH may be an important cause of LUTS and OAB. LUTS and OAB may be caused by multiple factors, and are not limited to males only, and have its own definition and classification. With BPH progression, obstructive symptoms during bladder emptying and irritation symptoms during bladder filling may occur alone or together. Obstructive symptoms are mainly caused by prostate enlargement-induced static pressure and dynamic factors of increased smooth muscle tension. Some studies showed that α₁ₐ adrenoceptor play an important role in mediating smooth muscle contraction of prostate and bladder neck, thereby resulting in obstructive symptoms. Irritation symptoms during bladder filling mainly manifest as OAB. Investigations have shown that BOO and OAB coexist in about 45% of BPH patients, and with progressive obstruction, the excitability of al receptor on detrusor gets increased, which makes detrusor's response to sympathetic excitation reversed from a relaxation effect mediated by β-receptor to a contraction effect mediated by al receptor, causing an instability in detrusor contraction in BPH patients, thus resulting in an increased occurrence of OAB. [Knutson T, Edlund C, Fall M, et al. BPH with coexisting overactive bladder dysfunction—an everyday urological dilemma, Neurourol Urodyn, 2001, 20(3): 237]. Of particular relevance to this invention, LUTS often co-exit with hypertension in the middle-aged and elderly men. About 25% of Western population over 60 years old have concomitant BPH/LUTS and hypertension [Maruenda J, Bhatnagar V, Lowenthal D T. Hypertension in the elderly with coexisting benign prostatic hyperplasia Urology, 1999, 53 (Suppl 3A): 7-12. Mc Vary K T. BPH: Epidemiology and comorbidities. Am J Manag Care. 2006 April, 12(5 Suppl): S122-8.]. Consistently, recent studies in China also showed that 30% of BPH/LUTS patients suffered from hypertension [Guo L, Zhang X, Li P. Na Y. A correlation study on benign prostatic hyperplasia and essential hypertension. Chinese Journal of Surgery 2005, (43)2: 108-111]. Although the underlying relation of hypertension to BPH/LUTS remains unclear, many data showed that hypertension and BPH/LUTS may interact with each other. 41% of hypertensive patients have more than seven IPSS (International Prostate Symptom Score) scores, while so do only 23% of non-hypertensive patients. The IPSSs of urination frequency and enuresis in hypertensive patients with BPH/LUTS are significantly higher than those in non-hypertensive patients with BPH/LUTS, and so is total IPSS [Shi X, Shi J, Ning X, et al. A study on relationship between benign prostatic hyperplasia and hypertension in the elderly in the rural area. Chinese Journal of Public Health, 2003, 19: 942-943; Torralba J A, Tornero Ruiz J, Banon Perez V, et al. Relation between hypertension and clinical cases of benign prostatic hyperplasia1 Arch Esp Urol, 2003, 56: 355-358]. In addition, it was also showed that an increase in diastolic blood pressure was associated with an earlier onset of BPH/LUTS and relevant surgical therapy. [Guo L, Zhang X, Li P, Na Y. A study on relationship between benign prostatic hyperplasia and essential hypertension. Chinese Journal of Surgery 2005, (43)2:108-111; Ning X, Shi J, Wu Z, et al. A Case-controlled study on risk factors for benign prostate hyperplasia in the population over 60 years old in the rural area in Shenyang. Chinese Journal of Epidemiology 2003, 24: 276-280.], and that the rate of prostate enlargement in volume correlates positively with diastolic blood pressure. [Hammarsten J, Hogstedt B. Hyperinsulinaemia as a risk factor for developing benign prostatic hyperplasia. Eur Urol, 2001, 39:151-158.].

Amlodipine is chemically known as 3-ethyLevo5-methyLevo2-(2-phthalimidoe thoxy) methyLevo4-(2-chlorophenyl)-1,4-dihydro-pyridine-6-methyLevo3,5-dicarboxylate. U.S. Pat. No. 4,572,909 disclosed Amlodipine and related dihydropyridine in compounds, which are potent anti-ischaemic and anti-hypertensive agents. U.S. Pat. Nos. 4,879,303 and 4572909 disclosed Amlodipine besylate and maleate, respectively. China patent No. 03164956 disclosed organic acid salt forms of Amlodipine. Amlodipine is a chiral compound, with levoamlodipine being of activity. (Arrowsmith, J. E; et al. Long-Acting Dihydropyridine Calcium Antagonists. 1,2-Alkoxymethel Derivatives Incorporating Basic Substituents. J. Med. Chem. 1986, 29; 1696-1702). Amlodipine, Levoamlodipine, Amlodipine besylate and other pharmaceutically acceptable salts of Amlodipine are potent and long-acting calcium channel blockers, and can be used as anti-hypertensive agents. Amlodipine besylate is currently for sale as NORVASC®, and levoamlodipine besylate as “Shihuida” on China's market.

### CONTENT OF THE INVENTION

The present invention provides a safe and effective treatment for patients with high IPSS scores, including irritating syndromes in bladder filling, and/or obstructive syndromes in bladder emptying, and low urinary flow rate, especially in patients with benign prostate hyperplasia, low urinary tract syndrome or overactive bladder.

Alpha receptor blocker is frequently used to control lower urinary tract disorders with concurrent hypertension, but it can not lower blood pressure effectively. Raising the level of the dosage of Alpha receptor blocker might lead to more side effects. Alpha receptor blocker is not the first choice for treating hypertension patient. Recurrence rate of hypertension was higher among patients with a long-term administration of Alpha receptor blocker. Solely control in the symptoms of lower urinary tract disorders or hypertension will not adequate to improve the quality of life for patients with lower urinary tract disorders with concurrent hypertension. Therapeutically strategies targeted to multifactors is in need. The invention provides a safer and more effective treatment for patients with both lower urinary tract disorders with concurrent hypertension.

### DETAILED DESCRIPTION OF THE PRESENT INVENTION

The present invention provides usages of composition comprising Amlodipine in the manufacture of medicaments for treating patients with benign prostate hyperplasia. Wherein said composition comprising active compounds and pharmaceutically acceptable cartiers or excipients. And said active compounds comprising Amlodipine, and pharmaceutically acceptable cartiers or excipients are the cartiers or excipient materials as well known in the field for preparing tablets, pill and capsules.

The usage of the present invention further provides usages of composition in manufacture of medicaments for treating benign prostate hyperplasia with concurrent hypertension. Amlodipine in above described usage is any one selected from the group consisting of Amlodipine, Amlodipine active metabolites, levoamlodipine, precursor of Amlodipine, pharmaceutically acceptable salts of precursor of Amlodipine, Amlodipine, Amlodipine active metabolites, levoamlodipine, or levoamlodipine active metabolites. Although the generic name of Amlodipine represents the free base, Amlodipine can also be used in the form of pharmaceutically acceptable salts. The pharmaceutically acceptable salts can be selected from the group of hydrochloride, sulfate, besylate, maleate, camphorsulfonic salt, salts of organic acids, and so on. As a common knowledge in the field, all salts of Amlodipine derived from the reaction between acid and hydrate are within the limits of the present invention. Amlodipine, in the present invention, is preferred to be Amlodipine Besylate and Levoamlodipine Besylate.

The pharmaceutical dosage range of Amlodipine in above described usage is from 1 mg to 10 mg, wherein 2.5 mg or 5 mg is preferred. The pharmaceutical dosage of Amlodipine active metabolites, levoamlodipine, precursor of Amlodipine, pharmaceutically acceptable salts of precursor of Amlodipine, Amlodipine, Amlodipine active metabolites, levoamlodipine, or levoamlodipine active metabolites can be calculated for corresponding pharmaceutical dosage range of Amlodipine by molecular weight.

Wherein said the benign prostate hyperplasia (BPH) is defined as benign hyperplasia in prostatic stroma, glands, connective tissues, and or smooth muscles; The above hyperplasia causes obstructive syndromes in bladder emptying and with or without irritating syndromes in bladder filling, whereas irritating syndromes include detrusor instability, bladder hyperesthesia, reduced bladder volume, urgent micturition, frequent micturition, urinary incontinence and nocturia. Obstructive symptoms in bladder emptying include dysuria, weak stream, terminal dribbling and incomplete emptying etc.

The usage of the present invention, wherein said the usage of composition for treating benign prostate hyperplasia with hypertension which defined as primary hypertension in accordance to the suggestive criteria of 1999 Guidance of Hypertension Prevention and Treatment in China and 1999 WHO/ISH Guidance of Hypertension Prevention and Treatment, diastolic blood pressure greater than 90 mmHg and or systolic blood pressure greater than 140 mmHg, after excluding secondary hypertension.

The present invention also provides usages of composition comprising Amlodipine in the manufacture of medicaments for treating low urinary tract syndromes. Wherein said composition comprising active compounds and pharmaceutically acceptable carriers or excipients. And said active compounds compose of Amlodipine, and pharmaceutically acceptable carriers or excipients are the carrier or excipient materials as well known in the field for preparing tablets, pill and capsules.

The usage of the present invention further provides usages of composition in manufacture of medicaments for treating lower urinary tract syndromes caused by benign prostate hyperplasia, especially lower urinary tract syndromes caused by benign prostate hyperplasia with concurrent hypertension.

Amlodipine in above described usage is any one selected from the group consisting of Amlodipine, Amlodipine active metabolites, levoamlodipine, precursor of Amlodipine, pharmaceutically acceptable salts of precursor of Amlodipine, Amlodipine, Amlodipine active metabolites, levoamlodipine, or levoamlodipine active metabolites. Although the generic name of Amlodipine represents the free base, Amlodipine can also be used in the form of pharmaceutically acceptable salts. The pharmaceutically acceptable salts can be selected from the group of hydrochloride, sulfate, besylate, maleate, camphorsulfonic salt, salts of organic acids, and so on. As common knowledge in the field, all salts of Amlodipine as a result of reaction between acid and hydrate are within the limits of the present invention. Amlodipine in the present invention is preferred to be Amlodipine Besylate and Levoamlodipine Besylate. The pharmaceutical dosage range of Amlodipine in above described usage is from 1 mg to 10 mg, wherein 2.5 mg or 5 mg is preferred. The pharmaceutical dosage of Amlodipine active metabolites, levoamlodipine, precursor of Amlodipine, pharmaceutically acceptable salts of precursor of Amlodipine, Amlodipine, Amlodipine active metabolites, levoamlodipine, or levoamlodipine active metabolites can be calculated for corresponding pharmaceutical dosage range of Amlodipine by molecular weight.

Wherein said the lower urinary tract syndromes (LUTS) is defined as a cluster symptoms in lower urinary tracts caused by multiple factors, including obstructive symptoms in bladder filling and/or irritating symptoms in bladder emptying, whereas irritating syndromes including detrusor instability, bladder hyperesthesia, reduced bladder volume, urgent micturition, frequent micturition, urinary incontinence and nocturia. Obstructive symptoms in bladder emptying include dysuria, weak stream, terminal dribbling and incomplete emptying etc.

Wherein the multiple factors include benign prostate hyperplasia, prostatitis, prostate cancer, bladder neck spasm, neurogenic neurological bladder, cerebrovascular diseases, bladder cancer, cerebrovascular diseases, Parkinson's disease, Alzheimer's and/or dementia, etc. Except for those specially refereed herein, the present invention specified the usages for treating lower urinary tract syndromes, not including the multiple factors which caused the syndromes.

The usage of the present invention, wherein said the usage of composition for treating lower urinary tract syndromes with concurrent hypertension which is defined as primary hypertension in accordance to the suggestive criteria of 1999 Guidance of Hypertension Prevention and Treatment in China and 1999 WHO/ISH Guidance of Hypertension Prevention and Treatment, diastolic blood pressure greater than 90 mmHg and or systolic blood pressure greater than 140 mmHg, after excluding secondary hypertension.

The present invention provides usages of composition in manufacture of medicaments for treating overactive bladder syndromes. Wherein said composition comprising active compounds and pharmaceutically acceptable cartiers or excipients. And said active compounds compose of Amlodipine, and pharmaceutically acceptable cartiers or excipients are the cartier or excipient materials as well known in the art for preparing tablets, pill and capsules.

The usage of the present invention further provides usages of composition in manufacture of medicaments for treating overactive bladder syndromes caused by benign prostate hyperplasia, especially overactive bladder syndromes caused by benign prostate hyperplasia with concurrent hypertension.

Amlodipine in above described usage is any one selected from the group consisting of Amlodipine, Amlodipine active metabolites, levoamlodipine, precursor of Amlodipine, pharmaceutically acceptable salts of precursor of Amlodipine, Amlodipine, Amlodipine active metabolites, levoamlodipine, or levoamlodipine active metabolites. Although the generic name of Amlodipine represents the free base, Amlodipine can also be used in the form of pharmaceutically acceptable salts. The pharmaceutically acceptable salts can be selected from the group of hydrochloride, sulfate, besylate, maleate, camphorsulfonic salt, salts of organic acids, and so on. As common knowledge in the field, all salts of Amlodipine as a result of reaction between acid and hydrate are within the limits of the present invention. Amlodipine in the present invention is preferred to be Amlodipine Besylate and Levoamlodipine Besylate. The pharmaceutical dosage range of Amlodipine in above described usage is from 1 mg to 10 mg, wherein 2.5 mg or 5 mg is preferred. The pharmaceutical dosage of Amlodipine active metabolites, levoamlodipine, precursor of Amlodipine, pharmaceutically acceptable salts of precursor of Amlodipine, Amlodipine, Amlodipine active metabolites, levoamlodipine, or levoamlodipine active metabolites can be calculated for corresponding pharmaceutical dosage range of Amlodipine by molecular weight.

Wherein said the Overactive bladder syndromes (OAB) is defined as primary overactive bladder syndromes or that caused by other relevant diseases, mainly a syndromes featured on irritating symptoms such as frequent micturition, urgent micturition, and urge urinary incontinence. Herein said overactive bladder caused by other relevant diseases includes benign prostate hyperplasia, female bladder neck obstruction, neurogenic bladder emptying dysfunction, partial lesion of bladder, impaired contractility of detrusor etc. Except for those specially refereed herein, our invention specified the usages for treating overactive bladder syndromes, not including the multifactors which caused the syndromes.

The usage of the present invention, wherein said the usage of composition for treating overactive bladder with hypertension which is defined as primary hypertension in accordance to the suggestive criteria of 1999 Guidance of Hypertension Prevention and Treatment in China and 1999 WHO/ISH Guidance of Hypertension Prevention and Treatment, diastolic blood pressure greater than 90 mmHg and or systolic blood pressure greater than 140 mmHg, after excluding secondary hypertension.

In the present invention, word “concurrent” or “company” means that a subject is suffering from two different disorders, such as lower urinary tract syndrome induced by benign prostate hyperplasia and hypertension. In fact, in our large-scale epidemiological investigation, we found that lower urinary tract disorders have intimate relationship with hypertension, and the two disorders often co-exist and affect each other, and especially, lower urinary tract disorders can be induced by or worsened by hypertension, and such inter-relationship is apparent when the lower urinary tract disorders are referred to be benign prostate hyperplasia, low urinary tract syndrome and overactive bladder. As a calcium channel blockers, Amlodipine has a diastolic smooth muscle effect, and Amlodipine can effectively act on lower urinary tract disorders described in this invention, but the mechanisms by which Amlodipine affect the relationship between low urinary tract disorders and hypertension remain to be studied. Thus, word “concurrent” or “company” means that Amlodipine can not only affect subjects suffering from lower urinary tract disorders or hypertension, but also the subjects suffering from both lower urinary tract disorders and hypertension, especially when lower urinary tract disorders are referred to be benign prostate hyperplasia, low urinary tract syndrome or overactive bladder, in which lower urinary tract syndrome or overactive bladder induced by benign prostate hyperplasia are preferred.

### THE BENEFIT OF THE PRESENT INVENTION

The present invention provides usages of composition comprising Amlodipine in the manufacture of medicaments for treating patients with high IPSS scores, including irritating syndromes in bladder filling, and/or obstructive syndromes in bladder emptying, and low urinary flow rate; especially in patients with benign prostate hyperplasia, low urinary tract syndrome or overactive bladder. It especially relates to treatment of lower urinary tract disorders with concurrent hypertension.

The present invention provides a safe and effective composition comprising Amlodipine in the manufacture of medicaments for treating patients with both lower urinary tract disorders and hypertension. It addresses an important clinical need.

In the present invention, the formulary form of said pharmaceutical composition includes, but not limit to tablets, double-layer tablets, multilayer tablets, sustained-release tablet, controlled-release tablets in single-chamber, dual-chamber controlled-release tablet, microporous controlled release tablet, sublingual tablet, rapidly disintegrating oral tablets, dispersible tablets, enteric-coated tablets, granules, pills, enteric-coated capsules, delayed-release tablets, from time to time/place-release tablets, capsules, sustained-release capsules, controlled-release capsule, capsule containing pellets or small pieces, pH-dependent capsule containing pellets or small pieces, oral liquid, film or paste form, etc. Particularly, the dosage form of said pharmaceutical composition is tablet or capsule.

In the present invention, the term cartier or excipients are the substances acting as the filling agent or cartier material in tablets, pill, capsule, etc, in the field. Generally these substances are approved by health administration institution to be used for the above purpose, and are pharmaceutically inactive, as described in <<Pharmaceutical expicients handbook>> (edited by A. Wade and P. J. Weller, the 2th edition, the United America Pharmaceutical institution, Washington and pharmaceutical publishing company, published in London, 1994). Especially, lactose, starch, cellulose ramification, etc, and their mixture could be used as the cartier for active substance of compound in the present invention.

In the present invention, pharmaceutically acceptable cartiers could be used in general oral preparation, including general tablets, general capsules, granules etc. Medicine cartiers in tablets include excipients and auxiliary substances that are used to prepare active compound to pharmaceutical agent, such as starch, microcrystalline cellulose, inorganic salt, sugar, dextrin, lactose, sugar powder, glucose, sodium chloride, cysteine, citric acid and sodium sulfite, etc, which could be used as single compound or as combination of several compounds, all of which are common knowledge in this field.

In the present invention, pharmaceutically acceptable cartiers could be used for sustained release preparation, including excipients and auxilury material etc. The excipients and auxilury materials include excipients reacting sustained release effects such as hydroxypropyl methylcellulose and/or ethyl cellulose and/or polyacrylic resins and/or carbomer and/or alginate-soluble/insoluble salt and/or ethyl cellulose and/or the other excipients as substained release excipients. Hydroxypropyl methylcellulose take kinds of commodities containing hydroxypropyl methylcellulose (HPMC) such as various strengths of Methocel, ethyl cellulose take kinds of commodities containing ethyl cellulose (EC), Polypropylene resins use polypropylene resins II [symbol] III or analog such as various strengths of propylene resins (Eudragit). The above excipients are pore-foaming agent, adhesive, lubricant, emulsifier, membrane materials, vesicant, bleaching auxiliaries, reagents or other excipients; Adhesive can use ethanol-water solution; Lubricant can use stearic acid, magnesium spar stearate, talcum powder, starch, paraffin etc; Solubilizer can use tartaric acid, citric acid etc; Emulsifizer can use span80\span85 etc; Membrane materials can use polyvinyl alcohol, hydroxyl methyl cellulose, hydroxyl ethyl cellulose, hydroxyethyl methyl cellulose, methyl cellulose etc; Pore-foaming agents use basic magnesium carbonate, sodium bicarbonate etc; Bleaching auxiliaries can use hexadecyl alcohol, octodecyl alcohol, beeswax etc; Reagents can use anhydrous alcohol, alcohol, water, etc.

In the present invention, pharmaceutically acceptable cartiers can be used for controlled release preparation, including active medicine and excipients operating controlled release action. The above excipients are polyoxyethylene and/or hydroxyl propyl cellulose and/or ethyl cellulose and/or sodium chloride and/or lactose and/or fructose and/or glucose and/or saccharose and/or low-substituted hydroxypropyl cellulose and/or crosslinked carboxymethyl cellulose sodium, and/or crosslinked polyvinylpyrrolidone and/or cellulose acetate. The above described excipients are medicine cartier, expandable materials, infiltration-aids, solubilizing agent, adhesive, wetting agents, lubricant, colorant agent, pore-foaming agent, membrane material, antisticking agent, plasticizer, opaquing agent, solvent. The medicine cartier, expandable materials can use polyoxyethylene, hydroxypropyl methyl cellulose, ethyl cellulose, behenic acid glycerides triglyceride etc; infiltration-aids can use sodium chloride, lactose, mannitol, fructose, glucose, sugar etc; solubilizing agent can use sodium dodecyl sulfate or poloxamer etc; Adhesive can use polyvinylpyrrolidone, hydroxypropyl methyl cellulose etc; Lubricant can use anhydrous alcohol, water, alcohol-water in various concentration; Lubricant can use stearic acid, magnesium stearate, starch, paraffin etc; Coloring agent can use iron oxide red, iron oxide yellow etc; Pore-foaming regent can use sugar, mannitol, polyethylene glycol, titanium dioxide, talcum powder, silicon dioxide etc; Membrane material can use cellulose acetate, ethyl cellulose etc; Solvent can use acetone, anhydrous alcohol, alcohol and water etc.

In the present invention, pharmaceutically acceptable cartiers can be used in sublingual tablet, fast disintegrating oral tablet or dispersible tablets, etc; including excipients and auxiliary material, etc. The above excipients and auxiliary material are low-substituted hydroxypropyl cellulose, microcrystalline cellulose, sodium carboxymethyl starch, crosslinked carboxymethyl cellulose sodium, and/or crosslinked polyvinylpyrrolidone, disposing of agar and mannitol, lactose etc.

In the present invention, pharmaceutically acceptable cartiers can be used in enteric-coated tablets and enteric-coated capsules etc; including excipients and auxiliary material etc. The above excipients and auxiliary material are starch, microcrystalline cellulose, inorganic salt, hydroxypropyl methyl cellulose, ethyl cellulose, polypropylene resins, carbomer, and/or alginate-soluble/insoluble salt, stearic alcohol, stearic acid, sugar, dextrin, sugar powder, glucose, sodium chloride, cysteine, citric acid and sodium sulfite etc one kind or several kinds of substance compound, enteric-coat materials includes: shellac, cellulose diacetate phthalates, acrylic resins (such as Eudragit L/S etc), polyvinyl acetate benzoates, phthalic acid hydroxypropyl methyl cellulose, succinic acid acetate hydroxypropyl methyl cellulose, polyvinyl acetate phthalates and plasticizer (such as diethyl phthalate, polyethylene glycol, propanediol, glycerol triacetate, dimethyl phthalate, dibutyl sebacate, (triethyl citrate, tributyl citrate, acetyl triethyl citrate, castor oil and various percentages of acetylated monoglyceride etc) and pore-foaming agents (such as PEG6000) etc kinds of excipients for pharmacy.

In the present invention, pharmaceutically acceptable cartiers can be used in delayed-release tablets or timed (position) release tablets, including excipients and auxiliary material etc. The above excipients and auxiliary material are starch, microcrystalline cellulose, inorganic salt, hydroxypropyl methyl cellulose, ethyl cellulose, polypropylene resins, carbomer, and/or alginate-soluble/insoluble salt, stearic alcohol, stearic acid, sugar, dextrin, sugar powder, glucose, sodium chloride, cysteine, citric acid and sodium sulfite etc one kind or several kinds of substance compound. The above coat materials operating delayed-release or timed(positon) release action include: shellac, cellulose diacetate phthalates, ethyl cellulose, hydroxypropyl methylcellulose, hydroxypropyl cellulose, acrylic resins, (such as Eudragit L/S etc) polyvinyl acetate benzoates, phthalic acid hydroxypropyl methyl cellulose, succinic acid acetate hydroxypropyl methyl cellulose, and plasticizer (such as diethyl phthalate, polyethylene glycol, propanediol, glycerol triacetate, dimethyl phthalate, dibutyl sebacate, triethyl citrate, tributyl citrate, acetyl triethyl citrate, castor oil and various percentages of acetylated monoglyceride etc) and pore-foaming agents (such as PEG6000) etc kinds of excipients for pharmacy.

In the present invention, pharmaceutically acceptable cartiers can be used in sustained release capsules, controlled release capsules, capsules containing pellets or flake, pH-reliance capsules containing pellets or flake, including excipients and auxiliary material. The above excipients and auxiliary material are starch, microcrystalline cellulose, inorganic salt, hydroxypropyl methyl cellulose, ethyl cellulose, polypropylene resins, carbomer, and/or alginate-soluble/insoluble salt, stearic alcohol, stearic acid, sugar, dextrin, sugar powder, glucose, sodium chloride, cysteine, citric acid and sodium sulfite etc one kind or several kinds of substance compound. The coat materials include: shellac, cellulose acetate phthalates esters, ethyl cellulose, hydroxypropyl methylcellulose, hydroxypropyl cellulose, acrylic resins (such as Eudragit L/S etc) polyvinyl acetate benzoates, phthalic acid hydroxypropyl methyl cellulose, succinic acid acetate hydroxypropyl methyl cellulose, and plasticizer (such as diethyl phthalate, polyethylene glycol, propanediol, glycerol triacetate, dimethyl phthalate, dibutyl sebacate, triethyl citrate, tributyl citrate, acetyl triethyl citrate, castor oil and various percentages of acetylated monoglyceride etc) and pore-foaming agents (such as PEG6000) etc kinds of excipients for pharmacy.

In the present invention, pharmaceutically acceptable cartiers can be used in granules, oral liquid, film agent, plaster etc. The pharmaceutically acceptable cartiers for preparation of film agent and plaster include excipients and auxiliary substance such as polyvinyl alcohol, cellulose triacetate, ethylene-vinyl acetate copolymer, polyvinylpyrrolidone, polyacrylamide, polyethy butylene material pressure sensitive adhesive, acrylic resin pressure sensitive adhesive, silicone pressure sensitive adhesive etc, and polyvinyl chloride, polyethylene, aluminum foil, polypropylene, polyester etc backing materials, polyethylene, polystyrene, polypropylene etc protective film, one kind or several kinds of substance compound.

### Example 1

### Effect Of Amlodipine On Benign Prostate Hyperplasia (BPH) in Rats

### Animal and Grouping

Male SD rats, in SPF grade, were randomly assigned into five groups: the blank (sham surgery, 30 rats), model (30 rats), Amlodipine #1 group (A1, 0.25 mg/kg, 15 rats), Amlodipine #2 group (A2, 0.5 mg/kg, 20 rats), or terazosin (T, 0.2 mg/kg, 20 rats, as a positive control) group. In addition, 20 spontaneous hypertensive rats (SHR) with prostate hyperplasia were taken as the Amlodipine #3 group (A3, 0.5 mg/kg).

### Establishment of Animal Model and Drug Administration

SD rats were anaesthetized by a peritoneal injection of 3.5% chloral hydrate at a dose of 350 mg/kg. Afterwards, the bilateral testicles of the rats were resected under an aseptic condition. Then the rats received an intramuscular injection of penicillin at 20000 U/kg/d for 3 days, and 1 week later a subcutaneous injection of testosterone propionate (dissolved in olive oil) at 0.5 mg/pec, once daily for 21 consecutive days.

In the treatment groups, the rats were orally administered by infusion at 1 ml/100 g body weight once daily for 14 consecutive days after 7-day injection with testosterone propionate. In the blank and model groups, the rats were orally perfused with isovolumetric physiological saline.

### Measurement of Intravesical Pressure & Urine Volume in Rats

The rats were anaesthetized by a peritoneal injection of 20% ethyl carbamate at a dose of 1000 mg/kg. The rat bladder was exposed by cutting a longitudinal incision on the upper of pubic arch. Piercing the top of bladder made a little hole, where a catheter with cannulas was inserted. The outside cannula (Φ 1.2 mm) with a pressreceptor was linked to PC-Lab multi-lead physiological recording system. The inside cannula (Φ 0.61 mm) was connected to an injection pump with constant infusion speed (2 ml/h). After last administration, the urodynamic parameters, single urine volume, and residual urine volume in rats were respectively collected.

### Statistical Methods

Data were presented as mean±SD. One way-ANOVA was employed for group comparison.

### Results

1. Effects on the Time and Interval of Micturition and on Average Urinary Flow Rate (Qₐᵥₑ) in Rats with BPH

The rats in the model group had a delayed time of micturition, a dramatically shortened interval of micturition, and a markedly decreased Qₐᵥₑ when compared to those in the blank group. The rats in A1 and A2 groups had a shorter time of micturition, a longer interval of micturition, and an elevated Qₐᵥₑ when compared to those in the model group. The rats in T group had also a shortened time of micturition, a markedly elevated Qₐᵥₑ, and a trend towards elongation of micturition interval. As compared to A1 and A2 groups, A3 group showed the best improvement in all the parameters as shown in Table 1.

TABLE 1
Treatment effects on the time and interval of micturition
and Qₐᵥₑ in rats with BPH ( x̅ ± s)

| | | Time of | Interval of | |
|---|---|---|---|---|
| Group | n | micturition (s) | micturition (s) | Q_{ave.} (ml/s) |
| Blank | 26 | 7.7 ± 1.2** | 862.7 ± 350.2** | 4.2 ± 1.4** |
| Model | 26 | 11.8 ± 2.3 | 525.4 ± 199.9 | 2.4 ± 1.0 |
| A1 0.25 mg/kg | 15 | 8.9 ± 1.6** | 734.8 ± 210.5* | 3.7 ± 1.2** |
| A2 0.5 mg/kg | 17 | 8.4 ± 1.8** | 762.3 ± 274.8** | 4.0 ± 1.9** |
| A3 0.5 mg/kg | 15 | 8.0 ± 1.1** | 786.0 ± 237.5* | 4.4 ± 1.6* |
| T 0.2 mg/kg | 17 | 8.1 ± 1.8** | 670.5 ± 288.5 | 3.7 ± 1.3** |

Compared to the model group,
*P < 0.05,
**P < 0.01
A1 0.25 mg/kg: Amlodipine 0.25 mg/kg; A2 0.5 mg/kg: Amlodipine 0.5 mg/kg; A3 0.5 mg/kg: SHR, Amlodipine 0.5 mg/kg; T 0.2 mg/kg: Terazosin 0.2 mg/kg

2. Effects on Point Pressure of Micturition and Peak Value of Micturition Pressure in Rats with BPH

The rats in the model group had a markedly increased point pressure of micturition and peak value of micturition pressure when compared to those in the blank group. The rats in A2, A3 and T groups had a remarkably decreased point pressure of micturition when compared to those in the model group. Yet, A2 and A3 groups had a lower peak value of micturition pressure than T group. See Table 2.

TABLE 2
Effects on point pressure of micturition and peak value of micturition
pressure in rats with BPH( x̅ ± s)

| | | Point pressure of | Peak value of micturition |
|---|---|---|---|
| Group | n | micturition (mmHg) | pressure (mmHg) |
| Blank | 26 | 31.2 ± 3.3* | 34.1 ± 5.2**^{##} |
| Model | 26 | 33.6 ± 4.4 | 38.7 ± 4.9 |
| A1 0.25 mg/kg | 15 | 33.4 ± 3.3 | 37.5 ± 7.0 |
| A2 0.5 mg/kg | 17 | 30.6 ± 3.9* | 30.5 ± 5.5**^{##} |
| A3 0.5 mg/kg | 15 | 31.4 ± 3.6* | 31.8 ± 7.3** |
| T 0.2 mg/kg | 17 | 30.9 ± 3.1* | 37.5 ± 5.0 |

Note:
Compared to the model group,
*P < 0.05,
**P < 0.01;
Compared to T group,
^{##}P < 0.01.
A1 0.25 mg/kg: Amlodipine 0.25 mg/kg; A2 0.5 mg/kg: Amlodipine 0.5 mg/kg; A3 0.5 mg/kg: SHR, Amlodipine 0.5 mg/kg; T 0.2 mg/kg: Terazosin 0.2 mg/kg.

3. Effects on Single Urine Volume and Residual Urine Volume in Rats with BPH

The rats in the model group had a decreased single urine volume and increased residual urine volume when compared to those in the blank group. All the treatment groups had higher single urine volume compared to the model group (but not statistically significant). The rats in A2, A3 and T groups had a remarkably decreased residual urine volume when compared to those in the model group, See Table 3.

TABLE 3
Effects on single and residual urine volumes
in rats with BPH( x̅ ± s)

| | | Single urine | Residual urine |
|---|---|---|---|
| Group | n | volume (ml) | volume (ml) |
| Blank | 27 | 0.35 ± 0.11* | 0.20 ± 0.12** |
| Model | 28 | 0.28 ± 0.13 | 0.50 ± 0.25 |
| A1 0.25 mg/kg | 15 | 0.32 ± 0.12 | 0.38 ± 0.31 |
| A2 0.5 mg/kg | 17 | 0.32 ± 0.09 | 0.33 ± 0.26* |
| A3 0.5 mg/kg | 15 | 0.31 ± 0.15 | 0.28 ± 0.27** |
| T 0.2 mg/kg | 17 | 0.29 ± 0.10 | 0.24 ± 0.22** |

Note:
Compared to the model group,
*P < 0.05,
**P < 0.01
A1 0.25 mg/kg: Amlodipine 0.25 mg/kg_{;} A2 0.5 mg/kg: Amlodipine 0.5 mg/kg; A3 0.5 mg/kg: SHR, Amlodipine 0.5 mg/kg; T 0.2 mg/kg: Terazosin 0.2 mg/kg

As for these urodynamic parameters in the experiment, the shortening of micturition time, a reduction of micturition point pressure, an increase in single urine volume and a decrease in residual urine volume mainly reflect improvement on symptoms of bladder outflow obstruction (BOO) as the result of the treatments. The elongation of micturition interval indicates an inhibition on overactive smooth muscle of bladder by the drugs. A decrease in peak value of micturition pressure reflects both the above therapeutic benefits.

The results demonstrated that Amlodipine can improve micturition point pressure and peak value of micturition pressure in the rats with BPH, suggesting an effective relief in symptomatic obstruction and irritation caused by BPH. Specially, a markedly reduction of peak pressure of micturition by Amlodipine displays its effect on overactive bladder (OAB). In the rats with BPH, Amlodipine decreased the micturition time, remarkably elongated the micturition interval, and increased Qₐᵥₑ. Amlodipine was superior to Terazosin in elongating the micturition interval, suggesting a possible inhibition on overactive bladder and improvement on symptoms such as urinary frequency. Meanwhile, Amlodipine possesses some actions in increasing the single urine volume and decreasing the residual urine volume, which further shows its beneficial effect on symptomatic obstruction caused by BPH.

The data also shows a dose-response relationship for Amlodipine to improve the symptoms of urinary tract obstruction and irritation (reflected by urodynamic parameters) in BPH rats. That is, administration with 0.5 mg/kg Amlodipine was superior to that with 0.25 mg/kg for these improvements. In a comparison of urodynamic parameters, administration with 0.5 mg/kg Amlodipine was identical to or a little superior to that with 0.2 mg/kg Terazosin. In addition, A3 group with SHR model tended to excel over A2 group without SHR model, suggesting that Amlodipine has a better improvement on the symptoms of urinary tract obstruction and irritation when BPH is companied with hypertension. The mechanism of this action is unclear.

### Example 2

### Effects of Amlodipine on Patients with Low Urinary Tract Syndrome (LUTS) and with Mild to Moderate Hypertension

### Inclusion Criteria

1) Male, 50-75 years old. 2) 140-180 mmHg in systolic pressure and/or 90-120 mmHg in diastolic pressure. 3) International Prostate Symptom Score (IPSS)≧8 points

### Exclusion Criteria

1) Having a history of hypersensitivity to Amlodipine and Terazosin. 2) Known or suspicious secondary hypertension. 3) Prostatic cancer. 4) Presence of severe diseases of internal medicine. 5) Companied with severe or active cardio-/cerebro-blood supply insufficiency. 6) Heavy smoker and drinker or drug addiction. 7) Evident abnormities in laboratory testing or physical signs, which suggest severe diseases, according to the investigator's judgment, or which likely, affect an observation and evaluation on therapeutic effects or adverse reactions of drugs, according to clinical specialists' judgment. 8) Moderate to severe hypertension, failing to be well controlled or to permit a removal of other α-receptor antagonists.

3-day wash-out was given for the patients who are taking some drugs affecting blood pressure or functions of lower urinary tract. Except for the treatment drugs, other drugs of lowering or raising blood pressure (e.g. anti-hypertensive drug) and of affecting the functions of lower urinary tract are not permitted during the study.

### Drugs and Grouping

Amlodipine besylate tablets, manufactured by American Pfizer (Dalian) Pharmaceutical Company, Terazosin hydrochloride tablets, manufactured by England Abbott (Shanghai) Pharmaceutical Company. Storage, reservation and delivery of drugs were in accordance with Standard Operation Procedure (SOP). The eligible patients were randomly assigned into the Amlodipine 5 mg group (90 subjects) or Terazosin 2 mg group (62 subjects). Administration was orally given before meal (at about 7:30˜8:00 am) for 4 consecutive weeks in each group.

### Observation Indices

A standard questionnaire of international prostate symptoms score (IPSS) was used to evaluate lower urinary tract symptoms, as seen in Table 4. Obstructive symptoms score includes hesitancy of micturition, reduced or interrupted urinary stream and sensation of incomplete bladder emptying, which mainly reflect BOO. Irritative symptoms score refers to urinary urgency and frequency, and an increase in night micturition, reflecting OAB symptoms. In this study, a total score of IPSS is considered as primary index. Sub-scores on BOO and OAB were also considered.

TABLE 4
Standard questionnaire of International Prostate Symptoms Score (IPSS)

| Is there any following symptoms in | | | | About | | Almost |
|---|---|---|---|---|---|---|
| the last month? | No | ⅕ | <½ | ½ | >½ | always |
| 1. Having a sensation of incomplete | 0 | 1 | 2 | 3 | 4 | 5 |
| bladder emptying? | | | | | | |
| 2. Want another after one micturition | 0 | 1 | 2 | 3 | 4 | 5 |
| within 2 h? | | | | | | |
| 3. Repeated discontinuation and | 0 | 1 | 2 | 3 | 4 | 5 |
| continuation of micturition? | | | | | | |
| 4. Too urgent micturition to be | 0 | 1 | 2 | 3 | 4 | 5 |
| delayed? | | | | | | |
| 5. Feeling urine flow getting slender? | 0 | 1 | 2 | 3 | 4 | 5 |
| 6. Needing a great effort in | 0 | 1 | 2 | 3 | 4 | 5 |
| micturition? | | | | | | |
| 7. Having an increased frequency in | 0 | 1 | 2 | 3 | 4 | 5 |
| getting up to micturate at night? | | | | | | |
| IPSS total up = | | | | | | |

Score for Quality of Life (QOL)

| | | | | | | | |
|---|---|---|---|---|---|---|---|
| What do you think if | Very | Satisfied | Roughly | Just | Not | Distressed | Very |
| you will have to be with | satisfied | | satisfied | so-so | satisfied | | distressed |
| current symptoms of | 0 | 1 | 2 | 3 | 4 | 5 | 6 |
| urological system during | | | | | | | |
| the rest of your life? | | | | | | | |
| QOL total up = | | | | | | | |

### Statistics

All data, doubly entered using Epi-info 3.0 and presented as mean±standard deviation (SD), were analyzed using SAS 8.0. A paired t-test was employed to compare pre- and post-treatment scores, and a multiple regression analysis was further performed to adjust for important covariates. Difference was considered significant at P<0.05.

### Results

For the two treatment groups, all indices and their changes in week 0 and week 4 are presented in Table 5. The patients in the Terazosin group showed a remarkable improvement in IPSS, BOO-related score, maximum urinary flow rate (Qₘₐₓ) and Qₐᵥₑ, and a decrease in systolic blood pressure, but had no improvement in OAB-related score and diastolic blood pressure. The patients in the Amlodipine group showed a remarkable improvement in IPSS, BOO- and OAB-related scores and a decrease in both systolic and diastolic blood pressure, and only Qₘₐₓ and Qₐᵥₑ showed no improvement.

TABLE 5
Effects of Amlodipine and Terazosin on symptoms of lower
urinary tract symptoms and blood pressure (mean ± SD)

| | |
|---|---|
| T (Terazosin) | A (Amlodipine) |
| N = 62 | N = 90 |

Week 0

| | | |
|---|---|---|
| IPSS | 13.2 ± 4.4 | 16.4 ± 6.1 |
| OAB | 6.6 ± 3.0 | 7.3 ± 3.3 |
| BOO | 6.6 ± 3.5 | 9.1 ± 4.2 |
| Qmax, ml/s | 17.7 ± 7.0 | 17.3 ± 6.7 |
| Qave, ml/s | 8.9 ± 3.9 | 8.9 ± 4.1 |
| SBP, mmHg | 152.8 ± 15.9 | 155.4 ± 16.1 |
| DBP, mmHg | 86.6 ± 9.0 | 88.6 ± 8.9 |

Week 4

| | | |
|---|---|---|
| IPSS | 9.4 ± 5.9 | 11.1 ± 6.7 |
| OAB | 5.3 ± 2.8 | 5.5 ± 3.2 |
| BOO | 4.2 ± 4.3 | 5.8 ± 4.6 |
| Qmax, ml/s | 19.4 ± 7.4 | 17.7 ± 6.9 |
| Qave, ml/s | 9.9 ± 4.2 | 8.8 ± 3.8 |
| SBP, mmHg | 147.0 ± 17.8 | 140.9 ± 15.4 |
| DBP, mmHg | 84.5 ± 11.4 | 82.0 ± 8.9 |

Percentage of improvement at Week 4

| | | |
|---|---|---|
| IPSS | 28.1 ± 40.5** | 30.2 ± 39.7** |
| OAB | 7.7 ± 54.8 | 17.3 ± 49.3** |
| BOO | 32.2 ± 63.5** | 33.3 ± 53.2** |
| Qmax, ml/s | 12.8 ± 30.5** | 7.1 ± 41.1 |
| Qave, ml/s | 13.8 ± 25.9** | 5.0 ± 35.2 |
| SBP, mmHg | 3.3 ± 11.9* | 8.9 ± 9.6** |
| DBP, mmHg | 2.3 ± 10.1 | 7.2 ± 7.1** |

Note:
A self-comparison between pre- and post- treatments:
*P < 0.05,
**P < 0.01;
SBP: systolic blood pressure; DBP: diastolic blood pressure.

Using multiple regression analysis, it was found that percentages of systolic and diastolic pressure lowering in the Amlodipine group were significantly higher than those in Terazosin group after 4-week treatment (P<0.01). On improving the symptoms of lower urinary tract symptoms, the two groups had similar improvement in IPSS, Qₘₐₓ and Qₐᵥₑ, as seen in Table 6.

TABLE 6
A multiple regression analysis to compare the two treatment groups for all index.

| | Percentage of Improvement | | | | | | | | | |
|---|---|---|---|---|---|---|---|---|---|---|
| | (1) | (2) | (2)-(1) | | | | | | | |
| | Terazosin | Amlodipine | Crude | | | | Adjust# | | | |
| | (N = 62) | (N = 90) | β | SE | P | 95% CI | β | SE | P | 95% CI |
| IPSS | 28.15 ± 40.5 | 30.17 ± 39.7 | 2.0 | 6.6 | 0.758 | −10.8~14.9 | 1.4 | 6.4 | 0.827 | −11.1~13.8 |
| OAB | 7.7 ± 54.8 | 17.3 ± 49.3 | 9.6 | 9.1 | 0.293 | −8.3~27.5 | 8.6 | 9.1 | 0.343 | −9.2~26.4 |
| BOO | 32.2 ± 63.5 | 33.3 ± 53.2 | 1.1 | 11.1 | 0.919 | −20.5~22.8 | 0.3 | 11.0 | 0.977 | −21.2~21.9 |
| Qmax, ml/s | 12.8 ± 30.47 | 7.09 ± 41.10 | −5.7 | 6.1 | 0.347 | −17.7~6.2 | −5.0 | 6 | 0.404 | −16.9~6.8 |
| Qave, ml/s | 13.77 ± 25.86 | 5.04 ± 35.25 | −8.7 | 5.2 | 0.094 | −18.9~1.5 | −8.4 | 5.2 | 0.106 | −18.6~1.8 |
| SBP, mmHg | 3.28 ± 11.92 | 8.89 ± 9.61 | 5.6 | 1.7 | 0.001 | 2.2~9.0 | 5.7 | 1.7 | 0.001 | 2.3~9.1 |
| DBF, mmHg | 2.31 ± 10.12 | 7.22 ± 7.09 | 4.9 | 1.4 | <0.001 | 2.2~7.6 | 5.1 | 1.4 | <0.001 | 2.4~7.8 |

Note:
#models were adjusted with variants of age and BMI.
In summary, the results showed that administration with 5 mg Amlodipine is superior to administration with 2 mg Terazosin in lowering blood pressure. Amlodipine exerts a therapeutic effect on both the BOO- and OAB-related scores, while Terazosin affects only the BOO-related score. For BPH patients with hypertension, Amlodipine is better in simultaneously controlling hypertension and both BOO- and OAB- related symptoms caused by BPH, which consist of two major syndromes of LUTS. Therefore, Amlodipine can be used as a therapeutic agent for the treatment of BPH, LUTS or OAB, especially with a concomitant hypertension.

### Example 3

### Effects of Amlodipine on Symptoms of Lower Urinary Tract in Patients at Different Blood Pressure Levels

### Methods

50 subjects with hypertension and 50 subjects with high normtension were enrolled in the study. IPSS in these subjects were all more than 8 points. All subjects received an oral administration with 5 mg/d Amlodipine for 4 consecutive weeks. The observation indices and statistical methods employed in the study were same as in Executed example 2. The concepts of high normtension and hypertension refer to <<A prevention guidance for hypertension in 2006>>(see Table 7).

TABLE 7
Definition and classification for blood pressure (BP) levels

| Classification | SBP (mmHg) | DBP (mmHg) |
|---|---|---|
| Normtension | <120 | <80 |
| High normtension | 120~139 | 80~89 |
| Hypertension: | ≧140 | ≧90 |
| Stage I hypertension (slight) | 140~159 | 90~99 |
| Stage II hypertension (moderate) | 160~179 | 100~109 |
| Stage III hypertension (severe) | ≧180 | ≧110 |
| Simple systolic hypertension | ≧140 | <90 |

### Results

At the baseline, there was no difference between the high normtension group and the hypertension group (see Table 8). After 4-week administration with Amlodipine, both the groups had a pronounced improvement on IPSS and symptoms of lower urinary tract, just as in the example 2. See Table 9. Of note, the subjects with hypertension more dramatically improved in their IPSS and BOO-related symptoms compared to those with high normtension (all P<0.05). After adjustment for age and BMI, this effect persisted, suggesting that Amlodipine is efficacious in treating the patients with co-morbid LUTS and hypertension. (see Table 9 and 10)

TABLE 8
Characteristics of subjects with high normtension
versus those with hypertension at baseline

| | High normtension | Hypertension | P_value |
|---|---|---|---|
| N | N = 50 | N = 50 | t-test |
| AGE, yr | 60.5 ± 5.6 | 60.1 ± 6.5 | 0.777 |
| BMI | 20.7 ± 3.1 | 21.3 ± 2.5 | 0.296 |
| IPSS | 16.2 ± 6.5 | 16.6 ± 5.7 | 0.779 |
| BOO | 8.7 ± 4.0 | 9.5 ± 4.5 | 0.364 |
| OAB | 7.6 ± 3.6 | 7.0 ± 2.8 | 0.367 |
| Q_{MAX,} ml/s | 17.1 ± 6.5 | 17.5 ± 7.0 | 0.797 |
| Q_{AVE,} ml/s | 9.1 ± 4.3 | 8.7 ± 3.9 | 0.684 |
| SBP, mmHg | 133.6 ± 4.8 | 159.4 ± 14.1 | 0.000 |
| DBP, mmHg | 83.2 ± 5.4 | 89.6 ± 9.1 | 0.000 |

TABLE 9
Effects of Amlodipine on percentage of index change and inter-group
comparison of 5 subjects with high normtension versus those
with hypertension between baseline and Week 4

| | | | Inter-group |
|---|---|---|---|
| | High normtension | Hypertension | comparison |
| N | N = 48 | N = 48 | P_value |
| IPSS | 21.8 ± 41.8** | 39.7 ± 35.2** | 0.032 |
| BOO | 20.9 ± 58.6* | 48.0 ± 42.3** | 0.015 |
| OAB | 17.5 ± 38.6** | 18.2 ± 49.8* | 0.978 |
| Q_{MAX,} ml/s | 5.8 ± 44.8 | 8.2 ± 38.0 | 0.789 |
| Q_{AVE} ml/s | 4.8 ± 33.4 | 5.5 ± 39.0 | 0.973 |
| SBP, mmHg | 6.6 ± 10.2** | 11.5 ± 8.2** | 0.017 |
| DBP, mmHg | 5.8 ± 7.9** | 8.8 ± 5.7** | 0.039 |

Note:
*Self comparison, P < 0.05;
**Self comparison, P < 0.01.

TABLE 10
A multiple regression analysis for effects of Amlodipine
on percentage of index change between baseline and Week 4

| | Ratio of improvement | | | | | | | | |
|---|---|---|---|---|---|---|---|---|---|
| | | Crude | | | | Adjust# | | | |
| | N | β. | SE. | P. | % 95 CI. | β. | SE. | P. | % 95 CI. |
| IPSS | 48 | 17.9 | 8.1 | 0.028 | 2.0, 33.8* | 17.7 | 7.9 | 0.026 | 2.1, 33.2* |
| PAI | 48 | 27.1 | 11.0 | 0.014 | 5.5, 48.6* | 28.1 | 10.8 | 0.009 | 7.0, 49.3** |
| CHU | 48 | −0.3 | 10.4 | 0.977 | −20.6, 20.0 | −1.9 | 10.2 | 0.852 | −22.0, 18.2 |
| Q_{MAX} ml/s | 48 | −2.3 | 8.6 | 0.786 | −19.3, 14.6 | 0.1 | 8.3 | 0.994 | −16.2, 16.3 |
| Q_{AVE} ml/s | 48 | −0.3 | 7.5 | 0.973 | −15.0, 14.5 | 1.5 | 7.4 | 0.835 | −13.0, 16.0 |
| SBP, mmHg | 48 | 4.8 | 2 | 0.014 | 1.0, 8.6* | 4.6 | 2 | 0.019 | 0.7, 8.4* |
| DBF, mmHg | 48 | 3.0 | 1.5 | 0.038 | 0.2, 5.9* | 3.2 | 1.4 | 0.027 | 0.4, 6.0* |

Note:
#the models were adjusted with covariates of age and BMI.

### Example 4

### Effects of Amlodipine on Blood Pressure in Patients with Different IPSS Scores

### Methods

100 subjects with hypertension (SBP140˜180 mmHg and/or DBP 80˜120 mmHg) were randomly selected and divided into two groups: one with IPSS≧8 and the other with IPSS<8. All subjects received an oral administration with 5 mg/d Amlodipine for 4 consecutive weeks. The observation indices and measuring and statistical methods employed in the study were same as in Executed example 2.

### Results

Baseline data: the population with IPSS≧8 were older than those with IPSS<8, showing a statistically significant difference between the two groups. After treatment with Amlodipine, both groups had their SBP and DBP remarkably lowered. Of note, Amlodipine had better SBP-lowering effect and much better DBP-lowering effect in the subjects with IPSS≧8 than in those with IPSS<8. After adjustment for age and BMI, the above effect persisted, suggesting that Amlodipine is efficacious in lowering blood pressure in the patients with IPSS≧8 (see Table 11-13).

In summary, all the data showed that Amlodipine can dramatically improve the symptoms of lower urinary tract. Meanwhile, Amlodipine also exerts a superior action on blood pressure-lowering in the subjects with IPSS≧8. Together, the data suggest that Amlodipine is an optimum in treating co-morbid hypertension and lower urinary tract disorders (including BPH).

TABLE 11
Characteristic of subjects with IPSS greater than
eight versus those lower than eight at baseline

| | IPSS < 8 | IPSS >= 8 | P value |
|---|---|---|---|
| N | N = 54 | N = 46 | (T test) |
| AGE, year | 58.2 ± 5.8 | 61.0 ± 6.3 | 0.000 |
| BMI | 21.3 ± 3.0 | 21.8 ± 2.8 | 0.323 |
| SBP, mmHg | 157.7 ± 14.4 | 159.0 ± 14.6 | 0.312 |
| DBP, mmHg | 90.7 ± 9.9 | 89.5 ± 9.2 | 0.173 |
| IPSS | 3.5 ± 2.0 | 15.0 ± 5.4 | 0.000 |

TABLE 12
Effects of Amlodipine on blood pressure change rate and inter-group
comparison of subjects with IPSS greater than eight versus those
lower than eight between baseline data and Week 4

| | | IPSS < 8 | IPSS >= 8 | P value |
|---|---|---|---|---|
| SBP, mmHg | 54 | 13.4 ± 17.6** | 15.9 ± 16.1** | 0.096 |
| DBP, mmHg | 46 | 6.9 ± 8.7** | 8.6 ± 8.7** | 0.025 |

Note:
T test:
*Self comparison, P < 0.05;
**Self comparison, P < 0.01.

TABLE 13
A multivariate regression comparison on effects of Amlodipine
on blood pressure change rate between baseline data and Week 4

| | Ratio of improvement | | | | | | | | |
|---|---|---|---|---|---|---|---|---|---|
| | | Crude | | | | Adjust* | | | |
| | N | β. | SE. | P. | %95 CI.,. | β. | SE. | P. | %95 CI.,. |
| SBP, mmHg | 54 | 1.5 | 0.9 | 0.103 | −0.3, 3.3 | 1.3 | 0.9 | 0.171 | −0.6, 3.0 |
| DBF, mmHg | 46 | 1.7 | 0.8 | 0.024 | 0.2, 3.3* | 1.5 | 0.8 | 0.050 | −0.0, 3.0 |

Note:
#Models were adjusted with variants of age and BMI.

1-12. (canceled)

13. A method of treating a lower urinary tract disorder selected from the group consisting of benign prostate hyperplasia, lower urinary tract syndrome and overactive bladder, wherein the lower urinary tract disorder is concurrent with hypertension, comprising administering to an individual in need thereof an active agent comprising Amlodipine.

14. The method of claim 13, wherein the Amlodipine is selected from the group consisting of Amlodipine, Amlodipine active metabolites, levoamlodipine, a precursor of Amlodipine, pharmaceutically acceptable salts of a precursor of Amlodipine, Amlodipine active metabolites, and levoamlodipine active metabolites.

15. The method of claim 14, wherein the pharmaceutically acceptable salt of Amlodipine is at least one of Amlodipine Besylate and Levoamlodipine Besylate.

16. The method claimed in claim 13, wherein the Amlodipine is administered in a pharmaceutical dosage range of 1 mg to 10 mg a day.

17. The method claimed in claim 13, wherein the Amlodipine is administered in a pharmaceutical dosage of 2.5 mg a day.

18. The method claimed in claim 13, wherein the Amlodipine is administered in a pharmaceutical dosage of 5 mg a day.
